(19) United States
(12) Patent Application Publication
(10) Pub. No.: US 2023/0139248 A1
(43) Pub. Date: May 4, 2023

(54) DEVICE AND METHOD FOR ASSESSING, PREDICTING AND OPERATING USERS HEALTH IN REAL TIME

(71) Applicant: ATTN: VIHATI TECHNO HEALTH PRIVATE LIMITED, Hyderabad (IN)

(72) Inventors: NIRANJAN RAVURI, Hyderabad (IN); NIVEDITHA ENAGANTI, Hyderabad (IN)

(21) Appl. No.: 17/052,832

(22) PCT Filed: Jul. 17, 2018
(86) PCT No.: PCT/IB2018/055282

§ 371 Date: Nov. 4, 2020

(30) Foreign Application Priority Data
May 17, 2018 (IN) 201841018540

(51) Int. Cl. A61B 5/0205; A61B 5/282; A61B 5/26; A61B 5/1172; A61B 5/00; A61B 7/00; A61B 7/02; A61B 5/145

(52) U.S. Cl. CPC A61B 5/02055; A61B 5/282; A61B 5/26; A61B 5/1172; A61B 5/7445; A61B 5/6806; A61B 7/003; A61B 7/02; A61B 5/14542; A61B 5/0006; A61B 5/0008; A61B 5/021

(57) ABSTRACT

Exemplary embodiments of the present disclosure are directed towards a medical device for assessing, and predicting and operating the user's health by capturing the user's vital signs in real time. The medical device comprises a plurality of electrodes and a plurality of sensors positioned on various finger sheaths, wrist portions, and hand portions. The various finger sheaths, the wrist portions, and the hand portions are configured to allow the plurality of electrodes to detect a plurality of electrical potentials on different surfaces of a user's body parts and the plurality of sensors to collect vital signs on different surfaces of a user's body parts. at least one processing device configured to contact with the plurality of electrodes and the plurality of sensors, the plurality of electrodes and the plurality of sensors configured to transmit the detected plurality of electrical potentials and the plurality of vital signs from the different surfaces of the user's body parts to the processing device. The processing device configured to store the plurality of electrical potentials and the plurality of vital signs and process the detected plurality of electrical potentials and the plurality of vital signs to assess a user's health and an end user device configured to receive the plurality of processed electrical potentials and the plurality of vital signs form the processing device through a network.

### TECHNICAL FIELD

The present disclosure generally relates to the field of medical devices. More particularly, the present disclosure relates to a medical device and method for assessing, predicting and operating the user's health by capturing the user's history, vital signs and other data in real time.

### BACKGROUND

Health care providers offer services to the patients on daily basis. As users grow older, chronic conditions develop and fall ill to serious health conditions, and require more frequent access to the health care providers (e.g., doctors, nurses, hospitals etc.). According to the report of World Health Organization (WHO) heart stroke, lower lung respiration sounds and brain strokes have topped the charts in causing deaths. However, these deaths can be avoided if the symptoms are identified at an early stage. This failure in the healthcare system is due to lack of awareness among the patients, expensive medical services, and infrastructure. Some medical devices are available to diagnose the patients but those come with a lot of expenditure and infrastructure.

Various medical devices are known in the field of medical instrumentation for monitoring, recording, and reporting the user's vital signs. It is a challenge to work towards regular improvement in sustaining the user's health. The users communicating with the healthcare providers are facing difficulties in emergency situations. Unfortunately, there has been no cost-effective model or user-friendly solutions in place at present for monitoring the user's vital signs. Those devices cannot be used to monitor, as they cannot function without having the user alongside them. This constraint makes it almost impossible for the users to have regular health evaluations. Thus, paving the way for the increase of chronic disease and untimely deaths due to the same.

In the light of the aforementioned discussion, there exists a need for a system with novel methodologies that would overcome or ameliorate the above-mentioned disadvantages.

### BRIEF SUMMARY

The following presents a simplified summary of the disclosure in order to provide a basic understanding to the reader. This summary is not an extensive overview of the disclosure and it does not identify key/critical elements of the invention or delineate the scope of the invention. Its sole purpose is to present some concepts disclosed herein in a simplified form as a prelude to the more detailed description that is presented later.

A complete appreciation of the present invention and the scope thereof can be obtained from the accompanying drawings which are briefly summarized below and the following detailed description of the presently preferred embodiments.

Exemplary embodiments of the present disclosure are directed towards a medical device and method for assessing, and monitoring the user's health by capturing the user's vital signs in real time.

An objective of the present disclosure is directed towards measuring user's vital signs in real time.

Another objective of the present disclosure is directed towards monitoring the health of the users continuously without hindering their daily activities.

Another objective of the present disclosure is directed towards a medical device that gives an opportunity to save and improve the quality of life of those suffering from long-term chronic health conditions and contributes to the increased medical awareness of the individuals.

Another objective of the present disclosure is directed towards replacing gel based single usage electrodes by non-sticky multiple usage touch electrodes.

Another objective of the present disclosure is directed towards performing gestures to capture the electrical potentials and vital signs.

Another objective of the present disclosure is directed towards utilizing a charger of the wearable device as the ECG leads.

Another objective of the present disclosure is directed towards operating the medical device to secure the user like the seat belt for the user in the seat of a motor vehicle.

According to an exemplary aspect, the medical device comprises a plurality of electrodes and a plurality of sensors positioned on a various finger sheaths, the various finger sheaths configured to allow the plurality of electrodes to detect a plurality of electrical potentials on different surfaces of a user's body parts and the plurality of sensors to collect the plurality of vital signs on different surfaces of a user's body parts.

According to another exemplary aspect, the medical device further comprises at least one processing device configured to contact with the plurality of electrodes and the plurality of sensors.

According to another exemplary aspect, the plurality of electrodes and the plurality of sensors configured to transmit the detected plurality of electrical potentials and the plurality of vital signs from the different surfaces of the user's body parts to the at least one processing device and the at least one processing device configured to store the plurality of electrical potentials and process the detected plurality of electrical potentials and the plurality of vital signs to assess a user's health.

According to another exemplary aspect, the medical device further comprises at least one end user device configured to receive the plurality of processed electrical potentials and the plurality of vital signs from the at least one processing device through a network.

### BRIEF DESCRIPTION OF DRAWINGS

Other objects and advantages of the present invention will become apparent to those skilled in the art upon reading the following detailed description of the preferred embodiments, in conjunction with the accompanying drawings, wherein like reference numerals have been used to designate like elements, and wherein:

FIG. 1 is a block diagram depicting a device, according to exemplary embodiments of the present disclosure.

FIG. 2 is a diagram depicting one exemplary implementation of the wearable device 102 having a palm portion and dorsum portion with finger sheaths, in accordance with one or more embodiments.

FIG. 3 is a diagram depicting another exemplary implementation of the wearable device 102 having a palm portion and dorsum portion with finger sheaths, in accordance with one or more embodiments.

FIGS. 4A-4B are diagrams depicting another exemplary implementation of the wearable device 102 having a left-hand palm portion, a right-hand palm portion, a left-hand and right-hand dorsum portion with finger sheaths, in accordance with one or more embodiments.

FIG. 5A is an example diagram depicting a mannequin having the wearable device 102, in accordance with one or more exemplary embodiments.

FIGS. 5B-FIG. 5C are diagrams depicting other exemplary embodiments of the mannequin having the wearable device 102, in accordance with one or more exemplary embodiments.

FIG. 5D is a diagram depicting the elongated charging cable 508a shown in FIG. 5A, in accordance with one or more exemplary embodiments.

FIG. 6 is flow diagram, depicting the method for assessing, and predicting and operating the user's health by capturing and detecting the electrical potentials, the user's vital signs in real time, in accordance with one or more embodiments.

FIG. 7 is a flow diagram, depicting the method for assessing, and predicting and operating the user's heart by detecting the electrical potentials and vital signs in real time, in accordance with one or more embodiments.

FIG. 8 is a flow diagram, depicting the method for detecting the electrical potentials and vital signs, in accordance with one or more embodiments.

### DETAILED DESCRIPTION

It is to be understood that the present disclosure is not limited in its application to the details of construction and the arrangement of components set forth in the following description or illustrated in the drawings. The present disclosure is capable of other embodiments and of being practiced or of being carried out in various ways. Also, it is to be understood that the phraseology and terminology used herein is for the purpose of description and should not be regarded as limiting.

The use of “including”, “comprising” or “having” and variations thereof herein is meant to encompass the items listed thereafter and equivalents thereof as well as additional items. The terms “a” and “an” herein do not denote a limitation of quantity, but rather denote the presence of at least one of the referenced item. Further, the use of terms “first”, “second”, and “third”, and the like, herein do not denote any order, quantity, or importance, but rather are used to distinguish one element from another.

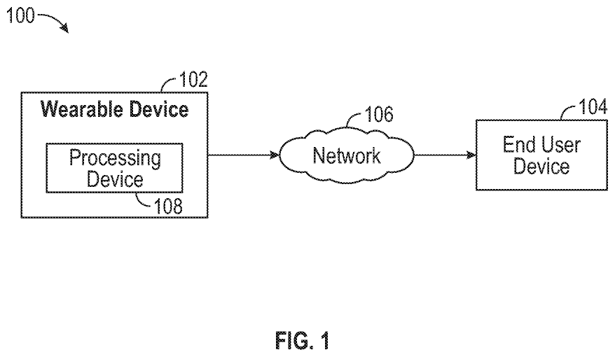

Referring to FIG. 1 is a block diagram 100, representing an environment in which aspects of the present disclosure can be implemented. Specifically, FIG. 1 depicts a schematic representation of a device for assessing, and predicting and operating the user's health in real time according to an embodiment of the present invention. The example environment is shown containing only representative devices and systems for illustration. However, real-world environments may contain more or fewer systems or devices. FIG. 1 depicts a wearable device 102, an end-user device 104, and a network 106. The wearable device 102 may be configured to detect and analyze the user's (e.g., the patient's) health in real time. The health may include, but are not limited to, user's vital signs (heart rate (e.g., heart sounds), respiration, blood pressure, temperature, oxygen saturation, breath sounds, intraocular pressure temperature), an electrocardiogram (EKG or ECGs), and other labs such as drug level, anemia urinalysis, bacteria in urine, bacteria in the blood, strep throat, ear infections, activities of the individual, body position, glucose levels, body weights index, ultraviolet radiation sensor, pollution sensor, pollen sensor, ultrasound probe, fatty liver recognition, galvanic skin response sensor, domestic gas sensor, hydration sensor, emotional levels through piloerection and sweating, tremor that makes it even a fitness tracker, calorie count GPS locator and the like. Here, the health may include, but is not limited to, the electrical potential.

The wearable device 102 may include but is not limited to, a medical glove, wearable apparatus, wearable sensors, wearable bands, a wearable watch, and the like. The end-user device 104 may include a system such as a server, a mobile phone, a personal computer, a workstation, a personal digital assistant, a mobile station, computing tablets, and the like. The network 106 may include, but is not limited to, an Ethernet, a wireless local area network (WLAN), or a wide area network (WAN), a Bluetooth low energy network, a ZigBee network, a WIFI communication network e.g., the wireless high speed internet, or a combination of networks, a cellular service such as a 4G (e.g., LTE, mobile WiMAX) or 5G cellular data service and IOT.

According to non-limiting exemplary embodiments of the present disclosure, the wearable device 102 may be worn on any one hand. The wearable device 102 may be configured to capture the user's health. The wearable device 102 may be configured to transmit the captured health data to the end user device 104 through the network 106. The health data may be recorded in the end-user device 104 by using the wearable device 102. The health data may be captured after contacting the user's body (e.g., the chest part) in a specific gesture by the wearable device 102. The wearable device 102 may comprise a processing device 108. The processing device 108 includes, but is not limited to, a microcontroller (for example ARM 7 or ARM 11), a microprocessor, a digital signal processor, a microcomputer, a field programmable gate array, a programmable logic device, a state machine or a logic circuitry.

The wearable device 102 may comprise electrodes (not shown) and sensors (not shown) positioned on various finger sheaths, wrist portions, and hand portions. The various finger sheaths, the wrist portions, and the hand portions may be configured to allow the electrodes (not shown) to detect electrical potentials on different surfaces of user's body parts and the sensors to collect vital signs on different surfaces of the user's body parts. The sensors (not shown) may be configured to detect the vital signs and transmit the detected vital signs to the end user device 104 through the network 106. The sensors (not shown) may include but are not limited to, a electrocardiograph (ECG) sensor, a pulse oximetry sensor, a phonocardiogram sensor, a temperature sensor, an emotion sensor, a hydration sensor, a blood pressure sensor, a respiratory sounds and patterns sensor, an angular and acceleration sensor, a fingerprint sensor, an electroencephalography (EEG) sensor, a glucometer, a ultrasound imaging sensor, an air quality and pollen sensor, a galvanic sensor, calorie count GPS locator, and the like. The end user device 104 may be configured to store the detected and analyzed health data of the user. The processing device 108 may be configured to store the electrical signals and other non-electrical signals and process the detected electrical potentials and other non-electrical signals and the collected vital signs to assess the user's health. The wearable device 102 which is gesture controlled using the electrodes (not shown) and collects the user's health information using the sensors (not shown).

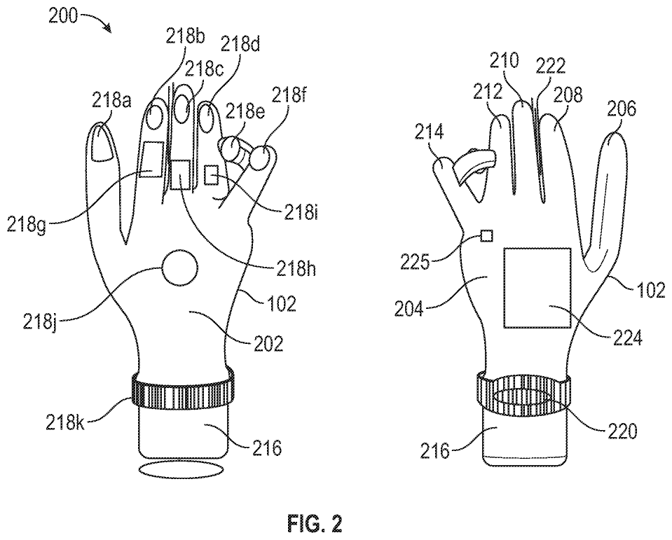

Referring to FIG. 2 is a diagram 200, depicting one exemplary implementation of the wearable device 102 having a palm portion and dorsum portion with finger sheaths, in accordance with one or more embodiments. The wearable device 102 may comprise a palm portion 202, a dorsum portion 204, a left thumb sheath 206, an left index finger sheath 208, a left middle finger sheath 210, a left ring finger sheath 212, a left little finger sheath 214 and a left hand wrist crease 216. The wearable device 102 may further comprise electrodes of 218a-218f placed at different positions of the body. The processing device 108 may be configured to take inputs from the sensors from the different positions of the body (e.g., left arm, right arm, leg foot, V1, V2, V3, V4, V5, and V6) and generate high-quality outputs. The user may wear the wearable device 102 on the left-hand. The wearable device 102 may comprise electrodes (e.g., V1, V2, V3, V4, V5, and V6) 218a-218f. The electrode 218a may be positioned on the thumb sheath 206, the electrode 218b may be positioned on the index finger sheath 208.

The electrodes 218a-218f may be configured to be electronically coupled to (e.g., detects the electrical activity) a heart of the user. The electrical potential detected by the electrodes 218a-218f may include directly detecting the electrical potential at the surface or indirectly detecting the electrical potential at the surface by detecting electrical characteristics of the surface that may be used to calculate the electrical potential. The calculated electrical potential may provide information about the electrical repolarization and depolarization of the heart during each heartbeat. In an example, the left thumb sheath 206 having the electrode 218a may be configured to contact the fourth intercostal space to the right of the sternum.

The left index finger sheath 208 having the electrode 218b configured to contact at the fourth intercostal space to the left of the sternum. The left ring finger sheath 212 having the electrode 218d configured to contact at the fifth intercostal space at a midclavicular line. The left middle finger sheath 210 having the electrode 218c configured to contact between the fourth intercostal space to the left of the sternum and the fifth intercostal space at the midclavicular line. A flexible joint having the electrode 218e made between the left ring finger sheath 212 and the left little finger sheath 214. The flexible joint may be configured to contact a left anterior axillary line.

The left little finger sheath having the electrode 218f may be configured to contact at the mid-axillary line at the same level as the electrode 218d and the electrode 218e. A finger (for e.g., right-hand finger) is drawn towards the top of the device or on to the edge of the extended left thumb sheath 206. A slot may be provided along with a sensor to receive the voltage from the right-hand. In an example, the left leg voltage can be taken by the specific gesture where the user can sit on a wooden chair or sofa fold his leg up and contact some part of the thigh with the elbow. Alternatively, there may be an elongated charging cable which may have a sensor embedded in the plug which can be attached to the left leg by placing it in the popliteal fossa, i.e., back of the knee joint and folding the leg for recording the left foot value. The right leg in contact with the floor completes the circuit as an earthing. The left middle finger sheath 210 further comprises an SPO2 and temperature sensor 218h configured to detect the temperature and the estimation of the oxygen saturation level usually measured with a pulse oximeter device.

The left ring finger sheath 212 further comprises a left arm sensor 218i. The left arm sensor 218i may be configured to detect the left arm voltage. The Phonocardiogram sensor (PCG) 218j may be positioned on the palm portion 202. The Phonocardiogram sensor (PCG) 218j may be configured to record the high-fidelity sounds and murmurs made by the heart and the breath sounds. The blood pressure sensor 218k may be positioned on the left hand wrist crease 216. The blood pressure sensor 218k may be configured to monitor the ambulatory blood pressure levels of the user. For e.g., the blood pressure sensor 218k monitors the ambulatory blood pressure levels for thirty days (30 days), 60 days and 90 days and so on. The blood pressure electrode 218k further comprises an air quality (p2.5 size particulate sensor) and pollen sensor 220. The air quality and pollen sensor 220 may also be configured to analyze the air quality of the surroundings. The ultrasound sensor 222 may be positioned between the index finger sheath 208 and the middle figure sheath 210. The ultrasound sensor 222 may be configured to capture the images and to diagnose any structural and functional disorders in the organs. The processing device 108 may be configured to record and assess the user's health and compare the existing data in the end user device 104. The Display unit and controls 224 may be positioned in the middle of the dorsum portion 204. The display and controls 224 may be configured to display the user's health data by capturing the user's vital signs in the real time. The sensor 218g may include an eye pressure sensor tonometer. The tonometer may be configured to measure intraocular pressure on a daily basis just with a gesture of touching the eyeball over the closed eye lid. Sensors 225 may be positioned on the dorsum portion 204. The sensors 225 may include but are not limited to, an ultraviolet radiation sensor, pollution sensor, pollen sensor, ultrasound probe, fatty liver recognition, domestic gas sensor, hydration sensor, emotional levels through piloerection and sweating, tremor, calorie count GPS locator, and the like.

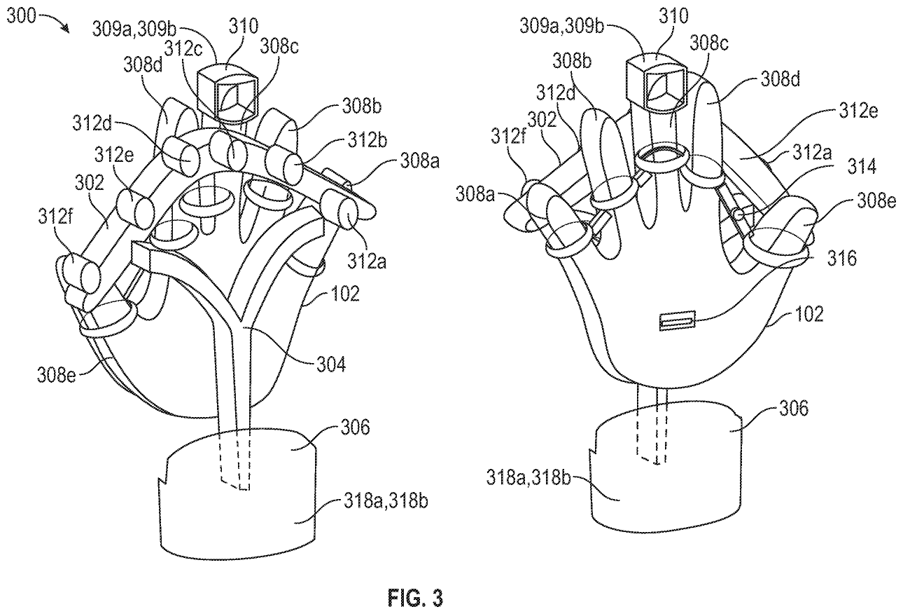

Referring to FIG. 3 is a diagram 300, depicting another exemplary implementation of the wearable device 102 having a palm portion and dorsum portion with left finger sheaths, in accordance with one or more embodiments. The wearable device 102 may comprise a signal acquisition unit which may have a flexible boomerang-shaped rod 302 supported via a “Y” shaped frame 304 and an elastic wristband 306. The “Y” shaped may be enabled the boomerang-shaped rod 302 to facilitate capturing signals. The flexible boomerang-shaped rod 302 may be worn over left finger sheaths 308a-308e to facilitate capturing signals across a proximal phalanx, a middle phalanx, and a distal phalanx equally. When the user wears the flexible boomerang-shaped rod 302 over the left finger sheaths 308a-308e, the various regions like: the boomerang-shaped rod 302 may be worn over the left finger sheaths 308a-308e with internal left finger sheaths touching the boomerang-shaped rod 302 and the external left finger sheaths touch the strap used to wear the boomerang-shaped rod 302. The middle finger 308c may have a vertical extension 310 from the boomerang-shaped rod 302, this vertical extension 310 acts as a cap to fix over the middle finger 308c. The vertical extension 310 may have a left arm sensor 309a configured to receive left-hand voltage. The vertical extension 310 may further comprise the SPO2 and temperature sensors 309b configured to detect the temperature and the estimation of the oxygen saturation level usually measured with a pulse oximeter device.

The region of boomerang-shaped rod 302 which the inner finger 308a-308e touches, the region of boomerang-shaped rod 302 which the outer finger 308a-308e touches, the region of boomerang-shaped rod 302 exterior to the region which the inner fingers 308a-308e touch, and the region of boomerang-shaped rod 302 exterior to the region which the outer fingers touch. These regions may be effectively used to capture the vital signs by using the region of boomerang-shaped rod 302 exterior to the region which the inner fingers touch. The boomerang-shaped rod 302 having the electrodes 312a-312f placed at different positions of the body. The electrical potential detected by the electrodes 312a-312f may include directly detecting the electrical potential at the surface or indirectly detecting the electrical potential at the surface by detecting electrical characteristics of the surface that may be used to calculate the electrical potential. The wearable device 102 may further comprise the right arm sensor 314, a USB port 316 the blood pressure sensor 318a, and the air quality and pollen sensor 318b. The right arm sensor 314 may be configured to detect the right arm. The blood pressure sensor 318a may be configured to monitor the ambulatory blood pressure levels of the user. The air quality and pollen sensor 318b may be configured to analyze the air quality of the surrounding. A USB charger that plugs into the USB port 316 to charge the wearable device 102. The elastic wristband 306 may be configured to secure around the wrist of the user for ensuring a secure comfortable fit. The elastic wristband 306 may be a blood pressure cuff. The index finger sheath 308d may be connected to the boomerang-shaped rod 302 having the electrode 312d (V3) and the electrode 312d (V3) midway between the electrode 312e (V2) and the electrode 312c (V4). The middle finger sheath 308c may be connected to the boomerang-shaped rod 302 having the electrode 312c (V4) configured to contact the fifth intercostal space at the midclavicular line. The boomerang-shaped rod 302 having the electrode 312e (V2) between the thumb sheath 308e and the index finger sheath 308d. The electrode 312e (V2) may be configured to contact the fourth intercostal space on the left of the sternum. The ring finger sheath 308b may be connected to the boomerang-shaped rod 302 having the electrode 312b (V5) configured to contact the anterior axillary line at the same level as the electrode 312c (V4). Here, the electrode 312b (V5) may be positioned under the breast in women. The little finger sheath 308a may be connected to the boomerang-shaped rod 302 having the electrode 312a (V6) configured to touch the midaxillary line at the same level as the electrode 312c (V4) and electrode 312b (V5). The thumb finger sheath 308e may be connected to the boomerang-shaped rod 302 having the electrode 312f (V1) configured to contact the fourth intercostal space, right of the sternum.

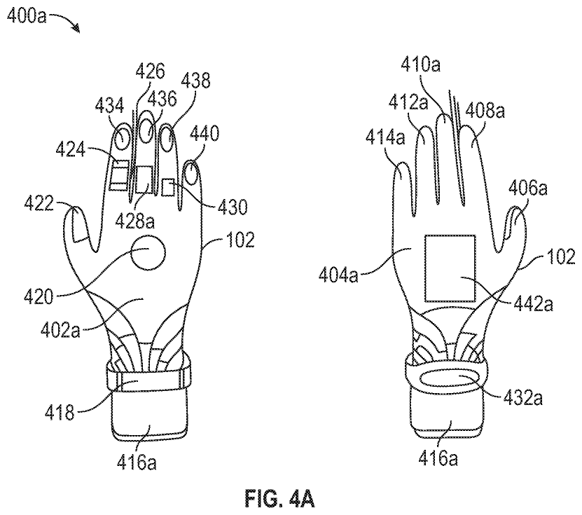

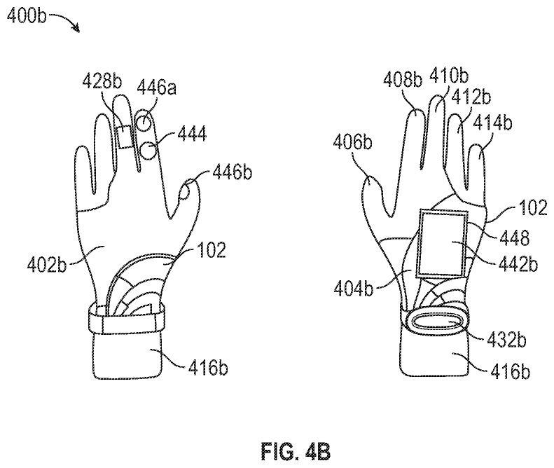

Referring to FIG. 4A-4B are diagrams 400a-400b, depicting another exemplary embodiment of the wearable device 102 having a left-hand glove and a right-hand glove, in accordance with one or more embodiments. The left-hand glove 400a depicts left-hand palm portion and a dorsum portion with finger sheaths. The FIG. 4A depicts the wearable device 102 which may comprise a left-hand palm portion 402a, a left-hand dorsum portion 404a, a left thumb finger sheath 406a, a left index finger sheath 408a, a left middle finger sheath 410a, a left ring finger sheaths 412a, a left little finger sheath 414a, a left-hand wrist crease 416a. The wearable device 102 may further comprises a blood pressure sensor 418 positioned on the left-hand wrist crease 416a, a phonocardiographic (PCG) sensor 420 positioned on the middle portion of the left-hand palm portion 402a, a fingerprint sensor 422 positioned on the top portion of the left thumb sheath 406a, an eye pressure sensor 424 positioned on the middle portion of the left index finger sheath 408a, an ultrasound sensor 426 positioned between the left index finger sheath 408a and the left middle finger sheath 410a, the SPO2 and temperature sensor 428a positioned on the middle portion of the left middle finger sheath 410a, a left voltage or potential sensor 430 positioned on the middle portion of the left ring finger sheath 412a, and an air quality and pollen sensor 432a positioned on the left-hand wrist crease 416a. The wearable device 102 may further comprise electrodes (v3, v4, v5, v6) 434-440 positioned on the top portion of the left index finger sheath 408a, the left middle finger sheath 410a, the left ring finger sheaths 412a, and the left little finger sheath 414a. The wearable device 102 may further comprise a Display unit and controls 442a may be positioned on the middle portion of the left-hand dorsum portion 404a.

The blood pressure sensor 418 may be positioned on the left-hand wrist crease 416a configured to monitor ambulatory blood pressure levels of the user. The phonocardiographic (PCG) sensor 420 may be configured to record the high-fidelity sounds and murmurs made by the heart and breath sounds. The fingerprint sensor 422 may be configured to collect the authentication of the user. The ultrasound sensor 426 may be configured to capture images and to diagnose any structural and disorders in the organs of the user's body. The ultrasound sensor 426 may be positioned above the knuckles for four fingers on the left-hand sheath or within a mechanical ridge provided between the index finger sheath 408a and the middle finger sheath 410a. In an example, a female user contacts her breast with the ultrasound sensor 426 to check for any swellings or abnormal growth and seeks surgeons' opinion may be obtained. The ultrasound sensor 426 may also be configured to perform a liver self-examination. Wherein the user can contact the surface around the liver to capture the structural images of the fatty liver and gallstones and any other growth in liver area. The ultrasound sensor 426 may further be configured to monitor fatty liver and gal stones and any other growth in the liver area.

As shown in FIG. 4B, the wearable device 102 may comprise a right-hand palm portion 402b, a right-hand dorsum portion 404b, a right thumb sheath 406b, a right index finger sheath 408b, a right middle finger sheath 410b, a right ring finger sheath 412b, a right little finger sheath 414b, and a right-hand wrist crease 416b. The wearable device 102 may further comprise a right arm sensor 444 positioned on the middle portion of the right index finger sheath 408b. The right arm sensor 444 may be configured to capture the right arm. The electrode 446b may be positioned on the right thumb sheath 406b touched to fourth intercostal space right of the sternum and the electrode 446a may be positioned on the right index finger sheath 408b touched to fourth inter costal space left of the sternum.

The electrodes (V1, V2) 446a-446b and the electrodes (V3, V4, V5, V6) 434-440 may be configured to detect the problems in the electrical activity of the head that may be associated with certain brain disorders. The gesture of keeping the hand over the head with same electrodes 434-440 and 446a, 446b nothing more. The SPO2 and temperature sensors 428b may be positioned on the middle portion of the right middle finger sheath 410b. The right-hand glove 400b may be worn whole day and the left-hand glove 400a may be stay at home. The right thumb finger sheath 406b and right index finger sheath 408b having the electrodes (V1, V2) 446a-446b and also the left finger sheaths 406a, 408a, 410a, 412a and 414a having the electrodes (V3, V4, V5, V6) 434-440 may be configured to place on the user's head to identify the electrical activity of the brain (e.g., electroencephalogram). The right thumb finger sheath 406b having the electrode 446b configured to contact at the fourth intercostal space to a right of the sternum of the heart and detect the electrical potentials from the fourth intercostal space. The right index finger sheath 408b having the electrode 446a configured to contact at the fourth intercostal space to the left of the sternum of the heart and detect the electrical potentials from the fourth intercostal space to the left of the sternum. The right index finger sheath 408b also having the right arm sensor 444 configured to capture the right arm. The left middle finger sheath 410a having the electrode 436 configured to contact the fifth intercostal space at the midclavicular line and detect the electrical potentials from the fifth intercostal space. The left middle finger sheath 410a also having the SPO2 and temperature sensor 428a configured to detect the temperature of the body and the blood oxygen levels. The left index finger sheath 408a having the electrode 434 configured to contact midway between the fourth intercostal space left of the sternum and the fifth intercostal space at the midclavicular line and detect the electrical potentials generated by heart from that angle. The left index finger sheath 408a further having the eye pressure sensor 424 configured to work as tonometer to measure intraocular pressure on a daily basis just with a gesture of touching the eye ball over the closed eye lid. The left little finger sheath 414a having the electrode 440 configured to contact at the midaxillary line at the same level as electrode 436 and 438 contact and detecting the electrical potentials of heart from the mid axillary line. The left ring finger sheath 412a having the electrode 438 configured to contact anterior axillary line at the same level as electrode 436 contact. The left ring finger sheath 412a having the voltage or potential sensor 430 configured to detect the voltage of the left-hand.

The SPO2 and temperature sensors 428a may be configured to detect the temperature and the estimation of the oxygen saturation level usually measured with a pulse oximeter device. The right-hand dorsum portion 404b further comprises a display unit and audio player 442b. The Display unit and audio player 442b may be configured to display the health data of the user and to motivate the user to achieve the set activity targets. The air quality and pollen sensor 432a may be positioned on the left-hand wrist crease 416a and the air quality and pollen sensor 432b positioned on the right wrist crease 416b. The air quality and pollen sensor 432a or 432b may be configured to analyze the air quality of the surrounding. The Display unit and audio player 442b may further include step and calorie count GPS locator 448. The step and calorie count GPS locator 448 may be configured to provide the GPS location of the user and upon the use of a panic button which may transmit the user locations to the end user device 104.

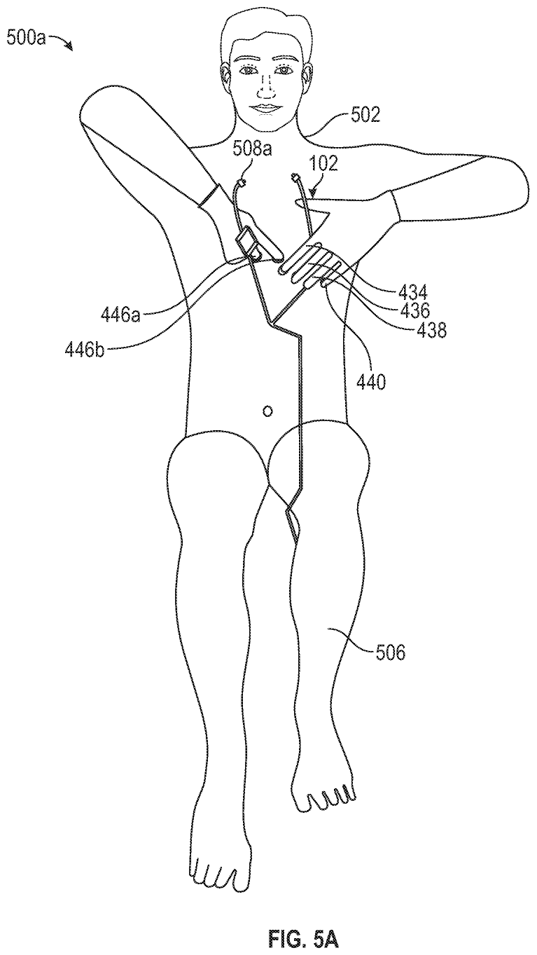

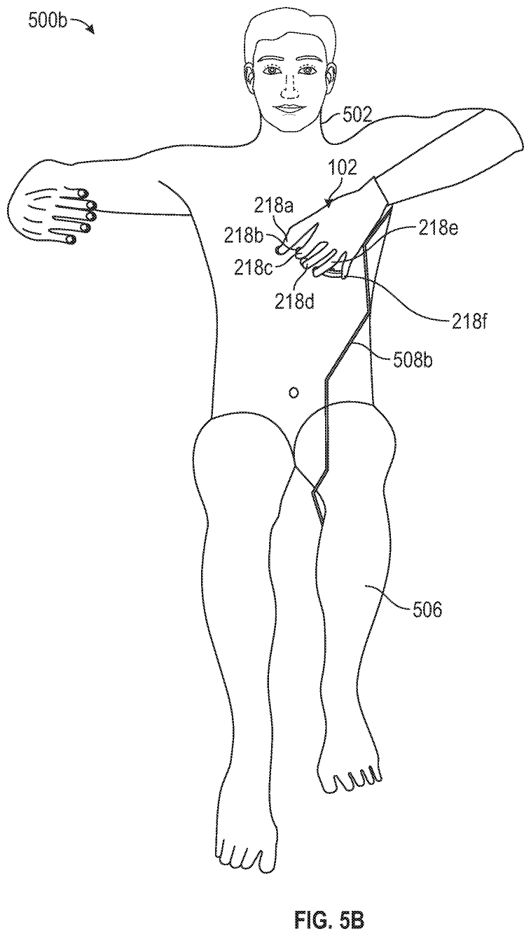

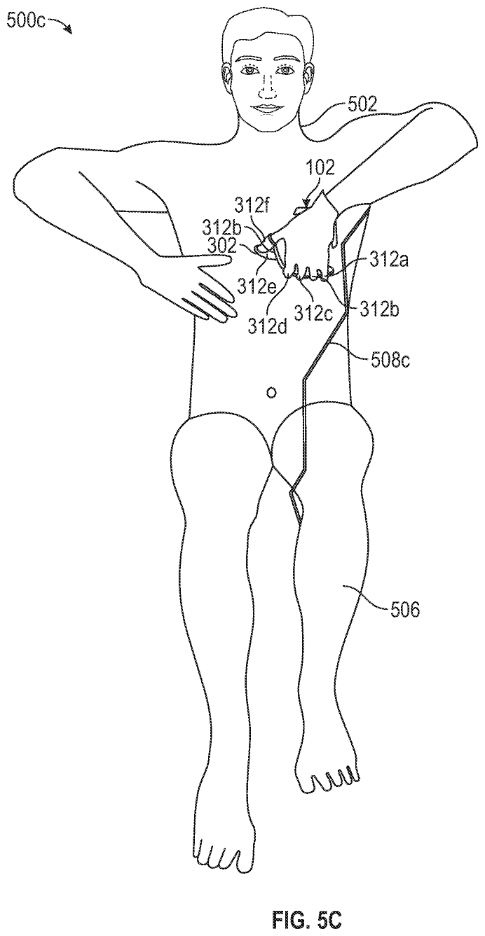

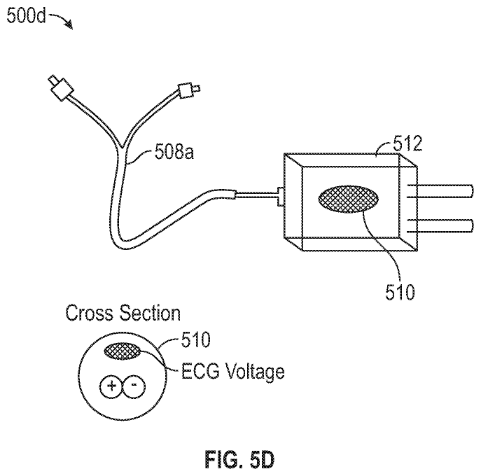

Referring to FIG. 5A is a diagram 500a depicting an exemplary embodiment of a mannequin having the wearable device 102, in accordance with one or more exemplary embodiments. The mannequin 502 having the wearable device 102 depicts the various finger gestures and/or finger movements. Gestures may be recognized by corresponding muscle activation even if a finger is missing. Furthermore, the gesture may be intended, measured, labeled, and/or classified. One or more types of intended gestures (e.g., curl, extend, tap, press hard, press light, or lift) may be combined with one or more different individual finger sheaths or groups of finger sheaths. The various finger sheaths having electrodes (V1, V2) 446a-446b (right-hand glove, for e.g.), electrodes (v3, v4, v5, v6) 434-440 (left-hand glove, for e.g.).The electrodes 446a-446b and 434-440 (e.g., V1, V2, V3, V4, V5, and V6) may be configured to capture the recordings from the mannequin (user's body parts, for e.g.) by just change in the gesture. The electrodes (V1, V2) 446a-446b (right-hand glove) and electrodes (v3, v4, v5, v6) 434-440 (left-hand glove) may also be configured to capture the electrical potentials and vital signs at a left leg 506. The diagram 500a further depicts an elongated charging cable 508a having a sensor (not shown) embedded in a plug (not shown) which may be attached to the left leg 506 by placing it in the popliteal fossa, i.e., back of the knee joint and folding the leg 506 for recording the left foot value. The elongated charging cable 508a may also be configured to connect the right-hand with the left glove to take the right-hand voltage when taking the ECG leads (12 leads, for e.g.). The right leg in contact with the floor completes the circuit as an earthing. The elongated charging cable 508a on one end may have the plug (not shown) which is embedded with a voltage sensor and on the other end branches into two USB cables 508a in the shape of “Y” which may attach to the wearable device 102 for charging (right and left glove respectively, for e.g.).

Referring to FIG. 5B-FIG. 5C are diagrams 500b-500c depicting other exemplary embodiments of the mannequin having the wearable device 102, in accordance with one or more exemplary embodiments. The diagram 500b depicting the mannequin 502 having the wearable device 102. The mannequin 502 having the various finger gestures and/or finger movements. The various finger sheaths having the various electrodes 218a-218f (e.g., V1, V2, V3, V4, V5, and V6). The electrodes 218a-218f (e.g., V1, V2, V3, V4, V5, and V6) may be configured to capture the recordings from the mannequin (user's body parts, for e.g.) by just change in the gesture.

The diagram 500c depicting the mannequin 502 having the wearable device 102. The mannequin 502 having the various finger gestures and/or finger movements. The various finger sheaths having the various electrodes 312a-312f (e.g., V1, V2, V3, V4, V5, and V6) and boomerang-shaped rod 302. The electrodes 312a-312f (e.g., V1, V2, V3, V4, V5, and V6) may be positioned on the boomerang-shaped rod 302. The electrodes 312a-312f (e.g., V1, V2, V3, V4, V5, and V6) may be configured to capture the recordings from the mannequin (user's body parts, for e.g.) by just change in the gesture.

The diagrams 500b-500c further depicts the charging cables 508b-508c embedded in a plug (not shown) which may be attached to the left leg 506 by placing it in the popliteal fossa, i.e., back of the knee joint and folding the leg 506 for recording the left foot value. The right leg in contact with the floor completes the circuit as an earthing. The charging cables 508b-508c on one end may have the plug (not shown) which is embedded with a voltage sensor and on the other end branches into the USB cables 508b-508c which may attach to the wearable device 102 (left gloves, for e.g.) respectively for charging.

Referring to FIG. 5D is a diagram 500d depicting the elongated charging cable 508a shown in FIG. 5A, in accordance with one or more exemplary embodiments. The elongated charging cable 508a having the sensor 510 embedded in the plug 512 which may be attached to the left leg 506 by placing it in the popliteal fossa, i.e., back of the knee joint and folding the leg 506 for recording the left foot value. The sensor 510 may include, but is not limited to, an ECG sensor. The elongated charging cable 508a on one end may have the plug 512 which is embedded with the voltage sensor and on the other end branches into two USB cables in the shape of “Y” which may attach to the wearable device 102 (right and left glove respectively, for e.g.) for charging. The diagram 500d further depicts the cross-section view of the sensor 510 (ECG sensor, for e.g.). The elongated charging cable 508a may be a single cable for the wearable device 102 (having left glove and right glove, for e.g.). The sensor 510 may be configured to capture the electrical potentials (the heart's electrical activity recorded from electrodes on the body surface, for e.g.) the elongated charging cable 508a have to be connected. The sensor 510 may also be configured to capture the electrical potentials at a left leg and the vital signs.

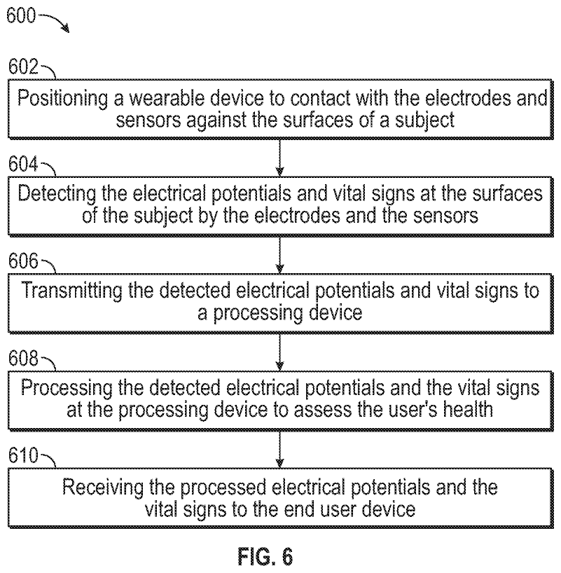

Referring to FIG. 6 is a flow diagram 600, depicting the method for assessing, and predicting and operating the user's health by detecting the electrical potentials capturing the user's vital signs in real time, in accordance with one or more embodiments. The method 600 may be carried out in the context of the details of FIG. 1, FIG. 2, FIG. 3, FIG. 4, and FIG. 5. However, the method 600 may also be carried out in any desired environment. Further, the aforementioned definitions may equally apply to the description below.

The method commences at step 602 where positioning the wearable device to have contact with the electrodes and the sensors against the surfaces of the subject. Here, the subject may include but is not limited to, the user's skin, user's head, user's chest, user's leg arms, user's hand arms, and the like. Thereafter, the method continues to next step 604 by detecting the electrical potentials and vital signs at the surface of the subject by the electrodes and the sensors. Thereafter, at step 606, transmitting the detected electrical potentials and vital signs to the processing device. Thereafter, at step 608, processing the detected electrical potentials and the vital signs at the processing device to assess the user's health. Thereafter, at step 610 receiving the processed electrical potentials and the vital signs to the end user device from the processing device.

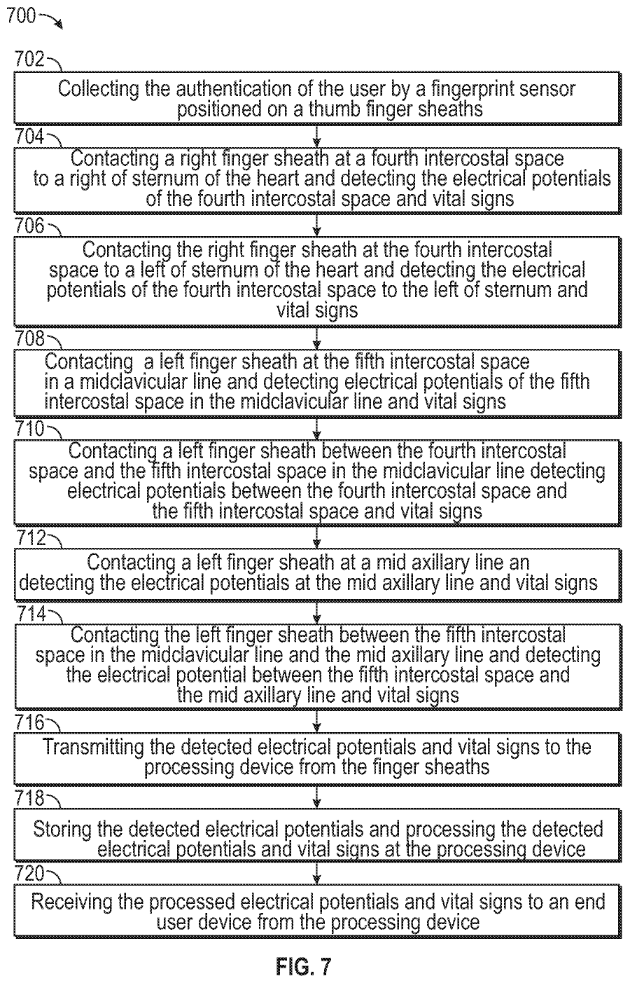

Referring to FIG. 7 is a flow diagram 700, depicting FIG. 7 is a flow diagram, depicting the method for assessing, and predicting and operating the user's heart by detecting the electrical potentials and vital signs in real time, in accordance with one or more embodiments. The method 700 may be carried out in the context of the details of FIG. 1, FIG. 2, FIG. 3, FIG. 4, FIG. 5 and FIG. 6. However, the method 700 may also be carried out in any desired environment. Further, the aforementioned definitions may equally apply to the description below.

The method commences at step 702 where collecting the authentication of the user by the fingerprint sensor positioned on the thumb finger sheath. Thereafter, at step 704, contacting the right finger sheath at the fourth intercostal space to a right of the sternum of the heart and detecting the electrical potentials from the fourth intercostal space and vital signs. Thereafter, at step 706, contacting the right finger sheath at the fourth intercostal space to the left of the sternum of the heart and detecting the electrical potentials from the fourth intercostal space to the left of the sternum and vital signs. Thereafter, at step 708, contacting the left finger sheath at the fifth intercostal space in midclavicular line and detecting electrical potentials from the fifth intercostal space in the midclavicular line and vital signs. Thereafter, at step 710, contacting the left finger sheath in the midway between the fourth intercostal space left of the sternum and the fifth intercostal space in midclavicular line and detecting electrical potentials. Thereafter, at step 712, contacting the left finger sheath at the midaxillary line and detecting the electrical potentials from the midaxillary line. Thereafter, at step 714, contacting the left finger sheath between the fifth intercostal space in midclavicular line and the midaxillary line and detecting the electrical potentials from there. In this process, capture various body vitals through the various sensors located within the embodiments and also capture the activity of the user and the ambient conditions where the user is present through these embodiments. Thereafter, 716, transmitting the detected electrical potentials and vital signs to the processing device. Thereafter, at step 718, storing the detected electrical potentials and vital signs and processing the detected electrical potentials and vital signs at the processing device. Thereafter, at step 720, receiving the processed electrical potentials and vital signs to the end user device from the processing device.

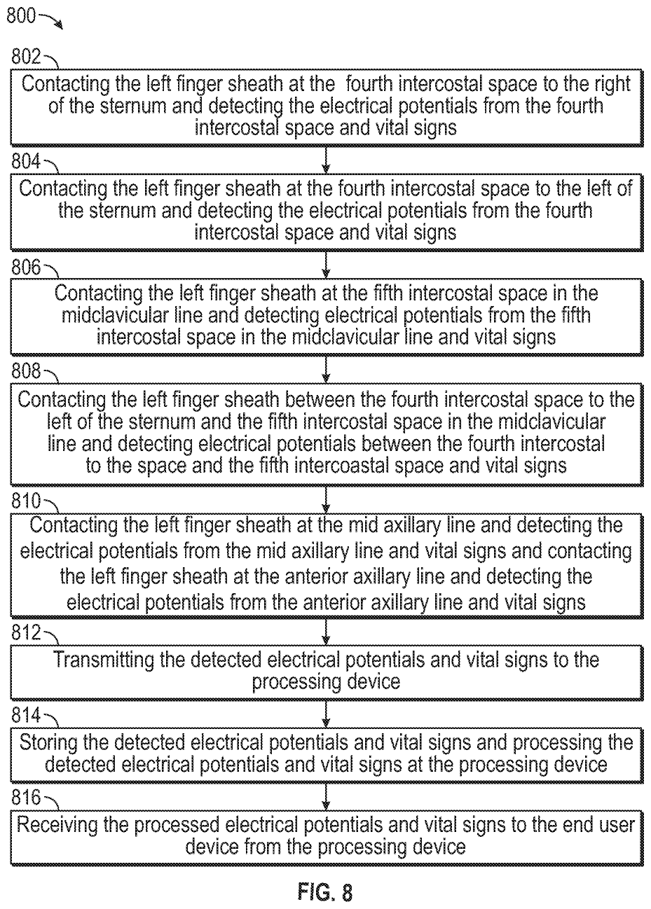

Referring to FIG. 8 is a flow diagram 800, depicting the method for detecting the electrical potentials and vital signs, in accordance with one or more embodiments. The method 800 may be carried out in the context of the details of FIG. 1, FIG. 2, FIG. 3, FIG. 4, FIG. 5, FIG. 6, and FIG. 7. However, the method 800 may also be carried out in any desired environment. Further, the aforementioned definitions may equally apply to the description below.

The method commences at step 802 contacting the left finger sheath at the fourth intercostal space to the right of the sternum and detecting the electrical potentials from the fourth intercostal space and vital signs. Thereafter, at step 804, contacting the left finger sheath at the fourth intercostal space to the left of the sternum and detecting the electrical potentials from the fourth intercostal space and vital signs. Thereafter, at step 806, contacting the left finger sheath at the fifth intercostal space in the midclavicular line and detecting electrical potentials from the fifth intercostal space in the nipple line and vital signs. Thereafter, at step 808, contacting the left finger sheath between the fourth intercostal space left of the sternum and the fifth intercostal space in the midclavicular line and detecting electrical potentials between the fourth intercostal space and the fifth intercostal space and vital signs. Thereafter, at step 810, contacting the left finger sheath at the mid axillary line and detecting the electrical potentials from the mid axillary line and vital signs and contacting the left finger sheath at the anterior axillary line and detecting the electrical potentials from the anterior axillary line and vital signs. Thereafter, 812, transmitting the detected electrical potentials and vital signs to the processing device. Thereafter, at step 814, storing the detected electrical potentials and vital signs and processing the detected electrical potentials and vital signs at the processing device. Thereafter, at step 816, receiving the processed electrical potentials and vital signs to the end user device from the processing device.

More illustrative information will now be set forth regarding various optional architectures and uses in which the foregoing method may or may not be implemented, as per the desires of the user. It should be strongly noted that the following information is set forth for illustrative purposes and should not be construed as limiting in any manner. Any of the following features may be optionally incorporated with or without the exclusion of other features described.

Although the present disclosure has been described in terms of certain preferred embodiments and illustrations thereof, other embodiments and modifications to preferred embodiments may be possible that are within the principles and spirit of the invention. The above descriptions and figures are therefore to be regarded as illustrative and not restrictive.

Thus the scope of the present disclosure is defined by the appended claims and includes both combinations and sub-combinations of the various features described hereinabove as well as variations and modifications thereof, which would occur to persons skilled in the art upon reading the foregoing description.

What is claimed is:

1. A device for assessing, and predicting and operating the user's health by capturing the user's vital signs in real time, comprising:
a plurality of electrodes and a plurality of sensors positioned on a various finger sheaths, wrist portions, and hand portions, whereby the various finger sheaths, the wrist portions, and the hand portions configured to allow the plurality of electrodes to detect a plurality of electrical potentials on different surfaces of a user's body parts and the plurality of sensors to collect a plurality of vital signs on different surfaces of a user's body parts;
at least one processing device configured to communicate with the plurality of electrodes and the plurality of sensors, whereby the plurality of electrodes and the plurality of sensors configured to transmit the detected plurality of electrical potentials and plurality of vital signs from the different surfaces of the user's body parts to the at least one processing device and at least one processing device configured to store the plurality of electrical potentials and the plurality of vital signs and process the detected plurality of electrical potentials and collected the plurality of vital signs to assess a user's health; and at least one end user device configured to receive the plurality of processed electrical potentials and the plurality of vital signs from the at least one processing device through a network.

2. The device of claim 1, wherein the plurality of sensors are configured to detect a blood oxygen saturation levels of the user and the plurality of sensors comprise a plurality of ultraviolet radiation sensors, a plurality of pollution sensors, a plurality of pollen sensors, a plurality of ultrasound probes, a plurality of domestic gas sensors, and a plurality of hydration sensors.

3. The device of claim 1, wherein the various finger sheaths are configured to capture the recordings from the user's body parts by just change in the gesture.

4. The device of claim 1, wherein the plurality of sensors are configured to monitor ambulatory blood pressure levels of the user, and plurality of sensors configured to capture the plurality of electrical potentials at a left arm and the plurality of vital signs, also the plurality of sensors configured to capture the plurality of electrical potentials at a right arm and the plurality of vital signs, and also the plurality of sensors configured to capture the plurality of electrical potentials at a left leg and the plurality of vital signs.

5. The device of claim 1, wherein the plurality of sensors configured to analyze the air quality of the surroundings of the different surfaces.

6. The device of claim 1, further comprising a plurality of display units positioned on a plurality of dorsum portions configured to display the user's health data.

7. A device for assessing and predicting and operating the user's health by capturing the user's vital signs in real time, comprising:
at least two palm portions and at least two dorsum portions comprising various finger sheaths, whereby the various finger sheaths comprising at least one finger sheath comprising at least one fingerprint sensor configured to collect an authentication of the user;
at least one right thumb finger sheath contacting a fourth intercostal space to a right of the sternum of a heart and the at least one right thumb finger sheath comprising at least one electrode configured to detect a plurality of electrical potentials at the fourth intercostal space to the right of the sternum and a plurality of vital signs, at least one right index finger sheath contacting the fourth intercostal space to a left of the sternum of the heart and the at least one right index finger sheath comprising at least one electrode configured to detect the plurality of electrical potentials at the fourth intercostal space to the left of the sternum and the plurality of vital signs;
at least one left middle finger sheath contacting the fifth intercostal space in midclavicular line and the at least one left middle finger sheath comprising at least one electrode configured to detect the plurality of electrical potentials at the fifth intercostal space in the midclavicular and the plurality of vital signs, at least one left index finger sheath contacting between the fourth intercostal space left of the sternum and the fifth intercostal space in the midclavicular line and the at least one left index finger sheath comprising at least one electrode configured to detect the plurality of electrical potentials between the fourth intercostal space and the fifth intercostal space and the plurality of vital signs, at least one left little finger sheath contacting a midaxillary line and at least one left little finger comprising at least one electrode configured to detect the plurality of electrical potentials and the plurality of vital signs at the midaxillary line, and at least one left ring finger sheath contacts between the fifth intercostal space and the midaxillary line and at least one left ring finger sheath comprising at least one electrode configured to detect the plurality of electrical potentials between the fifth intercostal space and the midaxillary line and the plurality of vital signs;
at least one processing device configured to process the detected electrical potentials and the plurality of vital signs; and
an end user device configured to receive the processed electrical potentials and the plurality of vital signs from the at least one processing device.

8. The device of claim 7, further comprising at least two right-hand finger sheaths comprising a plurality of electrodes configured to capture the plurality of electrical potentials at a right arm and the plurality of vital signs.

9. The device of claim 7, further comprising at least one left-hand sheath comprising at least one slot along with a sensor to receive a voltage from a right-hand sheath.

10. The device of claim 7, further comprising at least four left-hand finger sheaths comprising a plurality of sensors configured to capture the plurality of electrical potentials at a left arm and the plurality of vital signs and also the plurality of sensors configured to capture the plurality of electrical potentials at a right arm and the plurality of vital signs.

11. The device of claim 7, further comprising at least one elongated charging cable integrated with an ECG sensor embedded in a plug and the ECG sensor is configured to capture the plurality of electrical potentials and the plurality of vital signs.

12. The device of claim 11, wherein the plug is attached to the left leg by placing it in the popliteal fossa.

13. The device of claim 11, wherein the at least one elongated charging cable on one end have the plug which is embedded with the voltage sensor and on the other end branches into two USB cables in the shape of “Y” which is attached to the wearable device for charging.

14. The device of claim 7, further comprising a flexible joint having the electrode made between the at least one ring finger sheath and the at least one little finger sheath.

15. The device of claim 7, further comprising at least one finger sheath having an SPO2 and temperature sensor configured to detect the temperature and the estimation of the oxygen saturation level and the at least one finger sheath having a left arm sensor configured to detect the left arm voltage and the at least one finger sheath having a right arm sensor configured to detect the right arm voltage.

16. The device of claim 7, further comprising the at least one palm portion having a phonocardiogram sensor configured to record the high-fidelity sounds and murmurs made by the heart and breath sounds.

17. The device of claim 7, further comprising at least one hand wrist case having a blood pressure sensor configured to monitor the ambulatory blood pressure levels of the user, at least one air quality and pollen sensor configured to analyze the air quality of the surroundings and at least one ultrasound sensor configured to capture the images and to diagnose any structural and functional disorders in the organs, and the at least one sensor configured to capture the plurality of electrical potentials at a left leg and the plurality of vital signs.

18. The device of claim 7, further comprising at least one boomerang-shaped rod supported via at least one “Y” shaped frame and a wristband.

19. The device of claim 18, wherein at least one boomerang-shaped rod is worn over finger sheaths to facilitate capturing signals across a proximal phalanx, a middle phalanx, and a distal phalanx equally.

20. The device of claim 19, further comprising the at least one left thumb finger sheath connected to the at least one boomerang-shaped rod having the at least one electrode configured to contact the fourth intercostal space, right of the sternum and the at least one left thumb finger sheath having the at least one electrode configured to contact the right arm to capture the right arm voltage.

21. The device of claim 19, further comprising the at least one left index finger sheath connected to the at least one boomerang-shaped rod having the at least one electrode and the at least one electrode midway between the at least two electrodes.

22. The device of claim 19, further comprising the at least one boomerang-shaped rod having at least one electrode between the at least one left thumb finger sheath and the at least one left index finger sheath and the at least one electrode configured to contact the fourth intercostal space on the left of the sternum.

23. The device of claim 19, further comprising the at least one left middle finger sheath connected to the at least one boomerang-shaped rod having the at least one electrode configured to contact the fifth intercostal space in the midclavicular line and the at least one finger sheath having the left arm sensor configured to detect the left arm voltage and the at least one finger sheath having the right arm sensor configured to detect the right arm voltage and the at least one finger sheath having the at least one sensor configured to capture the plurality of electrical potentials at the left leg and the plurality of vital signs..

24. The device of claim 19, further comprising the at least one left little finger sheath connected to the at least one boomerang-shaped rod having the at least one electrode configured to contact the midaxillary line.

25. The device of claim 19, further comprising at least one left ring finger sheath connected to the at least one boomerang-shaped rod having the electrode configured to contact the anterior axillary line.

26. A method for assessing and predicting and operating the user's health by capturing the user's vital signs in real time, comprising:
positioning a wearable device to have contact with a plurality of electrodes and a plurality of sensors against surfaces of a subject;
detecting a plurality of electrical potentials of the surfaces and a plurality of vital signs of the surfaces of the subject by the plurality of electrodes and the plurality of sensors;
transmitting the detected plurality of electrical potentials and the vital signs to a processing device;
storing the detected plurality of electrical potentials and the plurality of vital signs and processing the detected plurality of electrical potentials and the plurality of vital signs at the processing device; and
receiving the processed plurality of electrical potentials and the plurality of vital signs to an end user device from the processing device.

27. The method of claim 26, further comprising a step of contacting at least one left finger sheath at a fourth intercostal space to the right of the sternum and detecting the plurality of electrical potentials from the fourth intercostal space and vital signs.

28. The method of claim 26, further comprising a step of contacting the at least one left finger sheath at the fourth intercostal space to the left of the sternum and detecting the plurality of electrical potentials from the fourth intercostal space and vital signs.

29. The method of claim 26, further comprising a step of contacting the at least one left finger sheath at the fifth intercostal space in the midclavicular line and detecting the plurality of electrical potentials from the fifth intercostal space in the in the midclavicular line and vital signs.

30. The method of claim 26, further comprising a step of contacting the left finger sheath between the fourth intercostal space to the left of the sternum and the fifth intercostal space and detecting the plurality of electrical potentials between the fourth intercostal space and the fifth intercostal space and vital signs.

31. The method of claim 26, further comprising a step of contacting the left finger sheath at the mid axillary line and detecting the plurality of electrical potentials from the mid axillary line and vital signs and contacting the left finger sheath at the anterior axillary line and detecting the plurality of electrical potentials from the anterior axillary line.
